(19) United States
(12) Patent Application Publication
(10) Pub. No.: US 2005/0187438 A1
(43) Pub. Date: Aug. 25, 2005

(54) Anti-stokes raman in vivo probe of analyte concentrations through the human nail

(75) Inventor: Jinchun Xie, Cupertino, CA (US)

Correspondence Address: SKYMOON RESEARCH & DEVELOPMENT, 3045 PARK BLVD., PALO ALTO, CA, 94306, US

(73) Assignee: Skymoon Research & Development, LLC

(21) Appl. No.: 10/787,909

(22) Filed: Feb. 24, 2004

(51) Int. Cl. A61B 5/00; A61B 6/00

(52) U.S. Cl. 600/310; 600/473

(57) ABSTRACT

A system and method are provided for detecting and quantifying an analyte in vivo. Anti-Stokes Raman scattered radiation emitted from a sample under incident radiation excitation is collected and analyzed. The intensity response is corrected for temperature effects using a Boltzmann correction factor based on the temperature of the sample. The sampled tissue is advantageously the sterile matrix beneath the nail of either a toe or a finger. The incident excitation radiation is projected onto the sterile matrix through the nail, which operates as a window. The present invention may be applied in both the blue/UV and the red/IR regions of the spectrum.

### BACKGROUND OF THE INVENTION

1. Related Applications

This application is related to co-pending U.S. patent application Ser. No. 10/723,042, filed on Nov. 26, 2003, the disclosure of which is incorporated herein by reference.

2. Field of the Invention

The present invention relates generally to the field of in vivo quantification of analytes in bodily tissues and/or fluids. More specifically, the present invention relates to the generation and detection of anti-Stokes Raman signals produced in the sterile matrix under the nail in different regions of the electromagnetic spectrum.

3. Brief Discussion of the Prior Art

Non-invasive body chemistry monitoring holds significant promise for a broad segment of the population. Approximately 16 million Americans and more than 100 million people worldwide who are afflicted with diabetes are advised to monitor their blood glucose levels several times each day. With currently available methods for measuring blood glucose levels, diabetics have blood drawn as many as five to seven times per day to adequately monitor their insulin requirements. Patients understandably do not enjoy having their blood drawn, and may avoid or delay glucose testing accordingly. Non-invasive, in vivo blood glucose measurement procedures may allow closer control of glucose levels without frequent, painful needle sticks, thereby substantially reducing the damage, impairment, and costs of diabetes. Other analytes of interest for which in vivo analysis techniques may be useful include, but are not limited to, urea, cholesterol, triglycerides, total protein, albumin, hemoglobin, hematocrit, and bilirubin.

Currently available optical measurement techniques for detecting and quantifying analytes in whole blood typically require calibration that involves blood draws and laboratory analysis. Available optical analysis techniques for whole blood are generally complicated by the low concentration of target analytes. The weak signals resulting from such low concentrations may be further distorted by absorption and scattering caused by red blood cells and/or other components of living tissue. In human tissue, the optical window is generally limited by water absorption features in the infrared (IR) region and by major bio-building blocks that absorb in the ultraviolet (UV) region of the spectrum. Specifically, protein and DNA have substantial absorption features in the UV spectral region due to amino acid and nucleic acid base groups. Overall, the window is limited from approximately the near UV to the near IR (NIR), as shown in FIG. 1. However, a number of chromophores add color to the tissue within this spectral window. This is especially true in three major body tissues: skin, blood, and muscle, which contain pigments, hemoglobin, and myoglobin, respectively. FIG. 1 shows the absorption spectrum of melanin, one of the predominant light absorbing species in skin pigmentation. As shown in FIG. 1, melanin has a strong absorption band in the UV region which decreases as a function of wavelength up to NIR. FIG. 1 also shows the absorption spectrum of hemoglobin. The red color of blood results from the strong hemoglobin absorption in the blue region of the spectrum. Myoglobin has a similar spectrum to hemoglobin, with strong absorption in the blue region.

Light scattering may be classified as elastic or inelastic scattering. Elastic scattering changes the direction of light propagation but not the light energy (i.e. the frequency or wavelength of the incident light). The causes of elastic scattering include rough surfaces or index mismatched particles as well as Rayleigh scattering from molecules. Inelastic scattering from matter changes the light energy (wavelength) as well as the propagation direction and polarization of the emitted photons relative to the incident photons, and is called Raman scattering. Raman scattering is a very powerful spectroscopic method for the detection of analytes, as the Raman spectra of different analytes are frequently more distinct than the spectra obtained by direct light absorption and/or reflectance.

Raman scattered radiation includes both anti-Stokes radiation generated at wavelengths shorter than the excitation light and Stokes radiation emitted at wavelengths longer than the excitation light. The Stokes signal results from a photonic interaction with a molecule in which the molecule absorbs energy and re-emits a lower energy scattered photon having a longer wavelength than the incident light. In contrast, anti-Stokes emissions result from a molecular relaxation to a lower energy state upon interaction with the incident photon. This energy is released as scattered photons with higher energy, and therefore lower wavelengths, than the incident exciting radiation.

Raman systems may be calibrated to provide information about absolute concentrations of analytes in a sample based on input data including the absolute scattering cross section, excitation laser path length, and photon collection efficiency from the sample interaction volume. These parameters are readily obtainable for transparent optical media in the gas phase or in solution. Human tissue, however, is a turbid media. Path lengths for the laser light passing through the tissue and the efficiency of the Raman scattering out of tissue are substantially more difficult to quantify. Thus, the use of Raman spectroscopy to quantify a specific analyte, such as glucose, in vivo is a challenging task.

Raman spectroscopic analysis of analytes in human tissues is further complicated by several additional obstacles. As noted above, human tissues have many absorption features that may attenuate the intensity both of incident excitation light into the tissue and of scattered light exiting the tissue. Additionally, certain tissues give off a fluorescence background upon laser excitation. This fluorescence may interfere with accurate quantification of the Raman signal by introducing a non-stable baseline. Similarly to the absorption curve of melanin shown in FIG. 1, fluorescence tends to be strongest at lower wavelengths, such as in the UV region. In general, as the excitation wavelength increases, the magnitude of the fluorescence response decreases. Additionally, fluorescence occurs at longer wavelengths (lower photon energy) than the incident light. Raman scattered light intensity is typically substantially weaker than the fluorescence response.

### SUMMARY OF THE INVENTION

The present invention provides systems and methods for analyzing the concentrations of one or more analytes in vivo using Raman spectroscopy.

In one embodiment, a method is provided for in vivo detection of an analyte. The method comprises the steps of illuminating a sample volume of body tissue with a beam of optical radiation having an incident wavelength from an optical source. Scattered anti-Stokes Raman radiation emitted within the sample volume is collected and then analyzed to determine an intensity response as a function of wavelength. The analyte concentration is then calculated based on the intensity response as a function of wavelength.

In an alternative embodiment, a system is provided for using anti-Stokes Raman spectography to detect an analyte in vivo. The system comprises a digit holder for positioning a digit. The digit comprises skin and a nail plate. The nail plate has a first end that is under the skin and a second opposite end that is disposed proximate to a tip of the digit. The digit holder comprises a substantially flat base plate that is attached to a back wall which is disposed approximately perpendicularly to the base plate such that the digit may be placed in the holder with a side of the digit opposite to the nail plate resting on the base plate and the second end of the nail plate disposed proximate to the back wall. The system further comprises a sensor attached to the digit holder for measuring the temperature of the digit and an incident light source that provides excitation radiation at an excitation wavelength. The excitation radiation is directed through the nail plate into a sterile matrix beneath the nail plate. A collection system for receiving scattered radiation emitted within the sterile matrix is also provided. This system may be adapted for use with either blue/UVA excitation radiation or red/IR excitation radiation. The temperature sensor may be adapted in concert with a dynamic feedback loop comprising a processor and a heating element to reactively stabilize the digit temperature in response to temperature measurements from the sensor.

In a further embodiment of the present invention, a method is provided for in vivo detection of an analyte. Te method comprises the steps of projecting excitation light onto a nail of a digit to illuminate a sample volume under the nail, measuring the temperature of the digit, and collecting Raman scattered light emitted from the sample volume. The Raman scattered light comprises an anti-Stokes signal. The Raman spectrum of the scattered light is processed to quantify one or more peak metrics for the anti-Stokes signal, and the peak metrics are corrected based on a Boltzmann correction factor that is calculated using the measured temperature of the digit. The analyte concentration is determined based on a partial least squares analysis using the Boltzmann-adjusted peak metrics.

### BRIEF DESCRIPTION OF THE DRAWINGS

Other objects and advantages of the present invention will become apparent upon reading the detailed description of the invention and the appended claims provided below, and upon reference to the drawings, in which:

FIG. 1 is a chart showing absorption curves of water, hemoglobin, diluted hemoglobin, melanin, protein, and DNA plotted on an exponential scale for both wavelength and absorbance.

FIG. 2 is a chart showing the approximate distribution of fluids in the human body.

FIG. 3 is a chart showing charge coupled detector (CCD) response curves from a back-illuminated CCD with peaks at 350 nm, 500 nm, and 850 nm.

FIG. 4 is a schematic diagram of a finger holder according to one embodiment of the present invention, that suppresses blood supply to the sterile matrix by pushing the fingernail against an L shaped stand in the horizontal direction.

FIG. 5 is a cartoon representation of a fingertip showing the contrast between the color intensity of a fingernail (a) with pressure applied at the end of the finger back toward the nail, (b) in its natural state with no pressure applied, and (c) with blood pooling resulting from pressure applied to the bottom and/or top of the fingertip.

FIG. 6 is a schematic diagram of a finger holder according to one embodiment of the present invention that enhances pooling of blood in the sampled sterile matrix by pushing the finger downward against a base.

FIG. 7 is a schematic diagram illustrating an anti-Stokes Raman probe system according to one embodiment of the present invention.

FIG. 8 is a schematic diagram illustrating an anti-Stokes Raman probe system according to an alternative embodiment of the present invention.

FIG. 9 is a flow chart describing the steps of a method for blood analyte analysis according to one embodiment of the present invention.

### DETAILED DESCRIPTION

The present invention provides a system and method for analyzing Raman measurements of analytes in tissue by measuring and quantifying scattered Stokes and/or anti-Stokes photons.

In general, according to the present invention, Raman scattered light emitted at either longer or shorter wavelengths compared to the exciting incident light may be collected and sent to a spectrograph. A fingernail may be used as a transparent window to reach the tissue containing analytes below, and to collect Raman light scattered from the sampled tissue. Alternatively, a toenail or some other anatomical feature with very low absorption characteristics for the incident light wavelength may be used. In the following description, “nail” generally refers to either a fingernail or a toenail.

As noted above, incident light that interacts with body tissue typically produces a fluorescence signal in the tissue in addition to whatever absorption and/or scattering interactions may occur. This fluorescence occurs at a longer wavelength (lower energy) than the pump light, and thus may overlap with and interfere with Stokes Raman emissions. Anti-Stokes scattering emissions occur at shorter wavelengths than the incident light. Thus, although anti-Stokes emissions generally have a substantially lower intensity than Stokes emissions, as discussed in greater detail below, avoidance of the varying baseline and other signal interference caused by fluorescence emissions may be quite beneficial.

According to one embodiment of the present invention, analyte concentration calculations are based on analysis of the anti-Stokes Raman signal instead of the Stokes signal. At anti-Stokes wavelengths, there is less overlap with fluorescence emissions from tissues because most fluorescence emissions occur at longer wavelengths than the excitation laser, not the shorter wavelengths where anti-Stokes signals occur. The present invention provides methods and systems for combining anti-Stokes Raman measurement and analysis into advantageous spectral windows for effective detection of analytes in human tissue in vivo.

According to a further embodiment of the present invention discussed in greater detail below, absorption of incident excitation light in the skin and muscles may be avoided through use of a nail as a “window” through which the incident laser light is projected to the sample. Glucose and other important blood analytes are equally concentrated in interstitial fluid and vascular fluid (blood). Accordingly, it is possible to reduce the impact of hemoglobin's strong absorption band by physically excluding blood in the tissue beneath the nail through exertion of gentle pressure on the nail. Use of a blue or UVA laser as the source of excitation light increases the Raman cross section dramatically while reducing absorption. In an alternative embodiment, anti-Stokes Raman emissions may be measured using a red or NIR laser as the incident light to further reduce the fluorescence background.

1. Absorption and Window in Human Tissues.

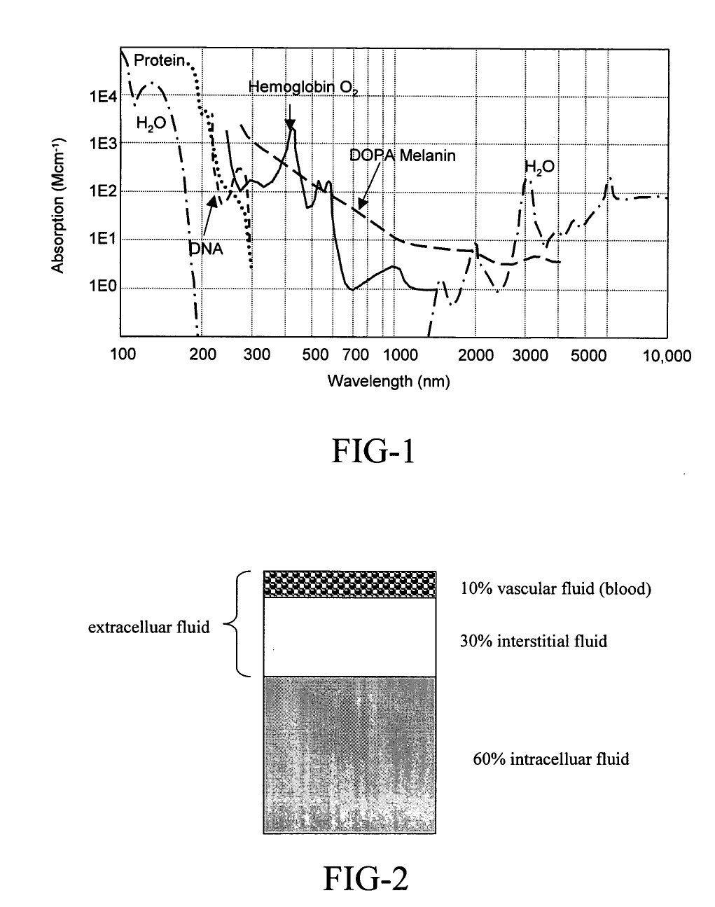

Strong absorption of incident and/or scattered light reduces the Raman signal of desired analytes. A system and method for Raman analysis of tissue that avoids these interferences is therefore quite desirable. To avoid strong absorption regions that may confound quantification of Raman scattered emissions, it is advantageous to use the red and NIR region of the spectrum in which most major elements show relatively low absorption, as shown in FIG. 1. In other spectral regions, it is advantageous to avoid wavelength bands in which colored skin, blood, and muscle may absorb more strongly. In comparison to skin, human finger and toe nails comprise mostly keratin and are therefore substantially transparent in the visible and NIR as well as UVA regions of the spectrum. Nail materials also do not contain the same chromophores as skin. These characteristics make nails a better window than skin in applications wherein light is used to probe analytes in blood and interstitial fluid. Furthermore, there are no muscles directly under the fingernail, so myoglobin absorption may be largely avoided. By manipulating the finger or toe through application of selective pressure, the blood contents under the nail may also be controlled to show red and white corresponding to pooling and suppressing blood as described in greater detail below.

FIG. 2 illustrates the approximate distribution of fluid in the human body. As shown, extracellular fluid, which accounts for approximately 40% of the total body fluid, is separated into 75% of interstitial fluid between cells and 25% of vascular fluid in blood. In general, glucose has the same concentration in interstitial fluid as in blood plasma in transcapillary tissue. Under the nail plate, there is sterile matrix tissue, which is filled with blood capillaries that provide adequate circulation for analytes in interstitial fluid and blood to ensure frequent equalization with concentrations throughout the body.

2. Fluorescence

Incident light with NIR wavelengths tends to induce relatively lower fluorescence response from human tissue than incident light in the UV and visible spectral regions. However, even at lower energy NIR wavelengths, the fluorescence background may present substantial problems in accurate quantification of the Raman Stokes signal. Fluorescence, like Raman Stokes emissions, occur at lower energy (long wavelengths) than the incident excitation light. Even in the NIR, fluorescence emissions may still be large enough to significant problems for quantification of the Raman Stokes signal. Anti-Stokes Raman emissions, which occur at lower wavelength than the excitation light, are not as significantly impacted by the fluorescence background because the fluorescence emission is at longer wavelength than the excitation light. Therefore, anti-Stokes Raman signal has little overlap with the fluorescence.

Due to the decrease in fluorescence intensity as a function of increasing wavelength, excitation wavelengths as long as approximately 1064 nm may facilitate accurate quantification of Stokes Raman emissions that are nearly free of fluorescence background. The primary disadvantage of using an excitation source at a wavelength greater than approximately 1064 nm is that the Raman spectrum may generally only be measured by a Fourier Transform (FT) system due to relatively low intensity Raman emissions. Typical silicon CCD arrays lack sufficient response at long wavelengths. In general, recording a useful spectrum at NIR wavelengths using FT requires long sampling times which are not consistent with the goal of in-vivo measurements on human subjects.

3. Anti-Stokes Raman Scattering

Anti-Stokes Raman emissions have an intrinsically weak signal compared to the Stokes signal because they originate from less populated vibrationally excited levels of molecules. As noted above, these emissions result from scattering of an incident photon accompanied by relaxation of the scattering molecule from an excited vibrational state. The population in a molecular vibrational level follows the Boltzmann distribution:
P(v)=e^{-v/kT} (1)
where v is vibrational energy, T is the temperature in K, and k is the Boltzmann constant. At human body temperature, approximately 310 K, a vibrational level at an energy of 1000 cm⁻¹ above the ground state contains approximately 1% of the molecular population of the ground state energy level. Thus, for such a vibrational state, the anti-Stokes Raman signal is 100 times weaker than the Stokes signal.

The Raman cross section of most molecules changes dramatically with the wavelength of the incident excitation light. The Stokes Raman relative cross section β, is
β=β₀v_{L}(v_{L}−v)³ (2)
while the anti-Stokes Raman relative cross section is
β=β₀v_{L}(v_{L}+v)³ (3)

where β₀ is the wavelength independent cross section, v_{L} is the inverse of the excitation wavelength (v_{L}=λ_{L}⁻¹) in wavenumbers, and v is the vibrational band in wavenumbers, which is much smaller than v_{L}. Because of the 4^{th} power dependency of β on v_{L}, the Raman cross section of a given molecule increases dramatically as the wavelength decreases. Table 1 summarizes Raman cross sections and Raman signal changes in a combination of six wavelengths and three vibration bands for glucose. The relative cross section values are normalized to the Raman band of 1130 cm⁻¹ at an excitation wavelength of 1064 nm excitation. The tabulated relative cross sections and overall signals are for a multiplying population at 310 K. In the blue and UVA regions of the spectrum, the Raman cross sections increase dramatically relative to those observed in the NIR.

TABLE 1
Relative Raman cross section of glucose and factored by population
at 310 K of body temperature for anti-Stokes and Stokes scattering
at six different wavelengths.

| | UVA | Blue | Red | NIR | | |
|---|---|---|---|---|---|---|
| Relative cross | 365 nm | 488 nm | 632.8 nm | 980 nm | Ref. I | Ref. II |
| section | Anti- | Anti- | Anti- | Anti- | 830 nm | 1064 nm |
| *population | Stokes | Stokes | Stokes | Stokes | Stokes | Stokes |
| β | 120 | 39.0 | 14.4 | 2.80 | 2.95 | 1.00 |
| β*P(1130 cm⁻¹) | 0.65 | 0.20 | 0.075 | 0.015 | 2.95 | 1.00 |
| β | 112 | 36.0 | 13.0 | 2.37 | | |
| β*P(524 cm⁻¹) | 10.5 | 3.4 | 1.13 | 0.21 | | |
| β | 111 | 35.4 | 12.8 | 2.32 | | |
| β*P(442 cm⁻¹) | 14.2 | 4.53 | 1.63 | 0.30 | | |

4. Temperature Variation and Stabilization

The strength of the anti-Stokes Raman signal is also sensitive to temperature as shown in equation 1. For Raman Stokes radiation, small temperature changes in a sample tend to have almost no impact on the spectrum intensity. Molecules that emit in the Stokes mode are mostly in the ground state. The relative number of ground state molecules in a given sample is not a strong function of temperature. In contrast, anti-Stokes radiation is emitted primarily from excited state molecules that relax back to the ground state upon interaction with an incident photon. The population of excited state molecules in a sample is much stronger function of temperature, so anti-Stokes signal strength is much more temperature dependent. The spectral peaks in the anti-Stokes spectrum exhibit stronger variations in a larger Raman shift, and weaker variation in a smaller shift.

The temperature of a fingertip or of a toe may fluctuate substantially from the core body temperature, and is dependent on factors such as environmental temperature variations, patient stress level, and the like. To address this issue, one embodiment of a Raman probe according to the present invention further comprises a sensor to monitor the temperature of the fingertip as it is pushed onto the finger stand. At the same time, the stand stabilizes the temperature of the finger. Anti-Stokes Raman measurements are advantageously not made until a stable finger temperature close to that of standard body temperature is reached and maintained. One of skill in the art may readily understand that a temperature sensor such as that described may also advantageously be incorporated into a sensor designed for the toenail and that such a system is also within the scope of the of the present invention as described herein. As noted above, the finger holders described herein my readily be modified by one of ordinary skill in the art for use as toe holders.

Measurement and maintenance of the sample temperature (such as for example the temperature of the finger or toe) at a stable, known value facilitates inclusion of the Boltzmann factor into a partial least squares (PLS) type multi-variate regression analysis program for improved calculation of analyte concentrations. Specifically, when using a multivariate technique to measure analyte concentrations, known spectra at a given concentration are required. Since the relative amplitudes of the components' spectra change with temperature, deviations of the sample temperature from that of the “calibration standard” may mimic a change in the relative concentrations of the analytes. Temperature changes may also alter the basis vectors such that the regression analysis will be unsuccessful. For example, in classical least squares (CLS), the relationship:
r=cS (4)
is employed, where r is the resulting total spectrum from the analytes (measured during an experiment), c is a vector containing the concentration of the analytes, and S is the matrix of measured spectra of each analyte (measured during calibration). The following linear algebra may be performed to determine the concentrations of each analyte:
r=cS (4)
rS^{t}=cSS¹ (5)
rS^{t}(SS^{t})⁻¹=cSS¹(SS^{t})⁻¹ (6)
c=rS¹ (7)
The superscripts “t” and “−1” in equations 5, 6, and 7 indicate the transposed matrix and inverse matrix, respectively. The predicted concentration in equation 7 relies on the fact that the spectra in S are known. The matrix S may be adjusted using Boltzmann corrections derived with measured temperature information and equation 1. One of skill in the art will note that the linear algebra procedure described herein is based on CLS. However, in PLS and other regression analysis routines according to various alternative embodiments of the present invention, CLS is a subset of the analysis. (“Chemometric techniques for quantitative analysis” Richard Kramer, Marcel Dekker, New York, 1998)
5. Windows for Anti-Stokes Raman Detection in Tissue.

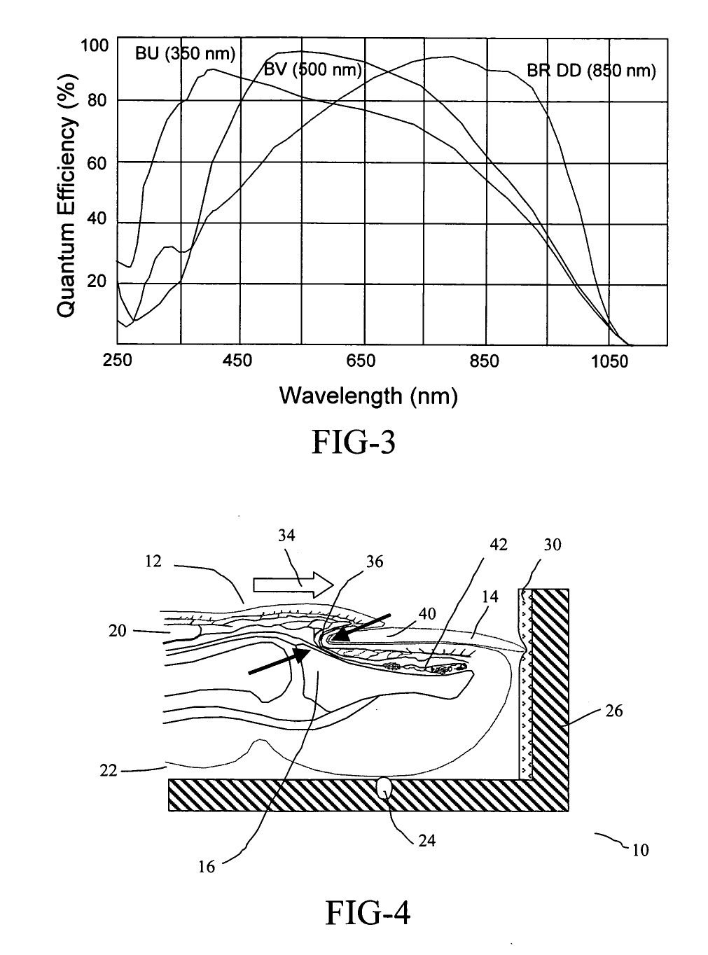

As discussed above in regards to FIG. 1, the absorption spectra of various tissue components provide a possible window for Raman detection. Table 2 summarizes various parameters of Stokes and anti-Stokes Raman emissions at several excitation wavelengths in this window. The hemoglobin peak at 406 nm (see FIG. 1) generally separates the available spectral window into two parts, one each in the UVA and blue regions. Use of the UVA window with an excitation wavelength of approximately 370 nm has the benefit of avoiding the strong absorption bands of DNA and protein at shorter wavelengths while also avoiding the main absorption peak of hemoglobin. The blue window lies in the “valley” centered at a wavelength of approximately 480 nm between the two hemoglobin absorption peaks shown in FIG. 1. At wavelengths longer than the second peak of hemoglobin at approximately 550 nm, the region from red to NIR presents an additional spectral window with very low absorption. In the NIR region, the window is practically limited by the sensitivity of currently available charge coupled device (CCD) detectors. The three major anti-Stokes bands resulting from excitation of glucose at a NIR wavelength of approximately 980 nm occur at approximately 882.3 nm, 932.13 nm and 939.31 nm, respectively. These wavelengths are close to the physical limit of currently available CCD detectors in the IR. In Raman measurements, a CCD offers numerous advantages including multiple channels of detection, high quantum efficiency, and extremely low noise. However, CCD response is a function of wavelength, and peak quantum efficiency typically occurs in the visible to very near infrared. Roll-off of CCD response occurs just below the visible region on the short wavelength side, and just above the visible on the long wavelength side. FIG. 3 shows response curves at three wavelengths for a typical CCD (Andor model number DU420, -BU, -BV, -BRDD, Southwindsor, CT06074).

TABLE 2
Raman bands of anti-Stokes and Stokes at 6 different
excitation wavelengths

| | UVA | Blue | Red | NIR | | Ref. II |
|---|---|---|---|---|---|---|
| | 365 nm | 488 nm | 632.8 nm | 980 nm | Ref. I | 1064 |
| | Anti- | Anti- | Anti- | Anti- | 830 nm | nm |
| Raman shift | Stokes | Stokes | Stokes | Stokes | Stokes | Stokes |
| 1130 cm⁻¹ | 350.54 | 462.50 | 590.57 | 882.30 | 915.90 | 1209.4 |
| 524 cm⁻¹ | 358.15 | 475.83 | 612.49 | 932.13 | | |
| 442 cm⁻¹ | 359.21 | 477.70 | 615.58 | 939.31 | | |
| CCD Q* | >80% | >80% | >90% | >80% | >80% | none |

*CCD Q stands for quantum efficiency for CCD detector. The numbers are quoted from Andor on back-illuminated CCD arrays detectors, BU(350 nm), BV(500 nm), BR(750 nm), and BR(850 nm).

6. Blue and UVA Embodiment

FIG. 4 shows a design for a finger holder 10 according to one embodiment of the present invention. The structure of a typical finger 12 includes the nail plate 14, the finger tip bone 16, blood vessels 20 that include arterial tissue and capillaries, and the skin 22. In general, the finger holder 10 may comprise a sensor 24 to measure finger temperature through contact with the surface 22 of the finger 12. In use, the nail plate 14 is pushed against the back wall 26 of the finger holder 10. For comfort, the back wall 26 may further comprise a padded surface 30 against which the fingertip may be pressed. When the nail plate 14 is pushed back along the main axis of the finger (shown by arrow 34), it suppresses the arterial vessels 36 lying in the narrow region behind the sub-cutanaceous end 40 of the nail plate 16 and the finger tip bone 20. As a result, the blood supply to the sterile matrix 42 under the fingernail plate is suppressed. This effect is visible on a typical human fingernail as a broad, pale or even “white” region.

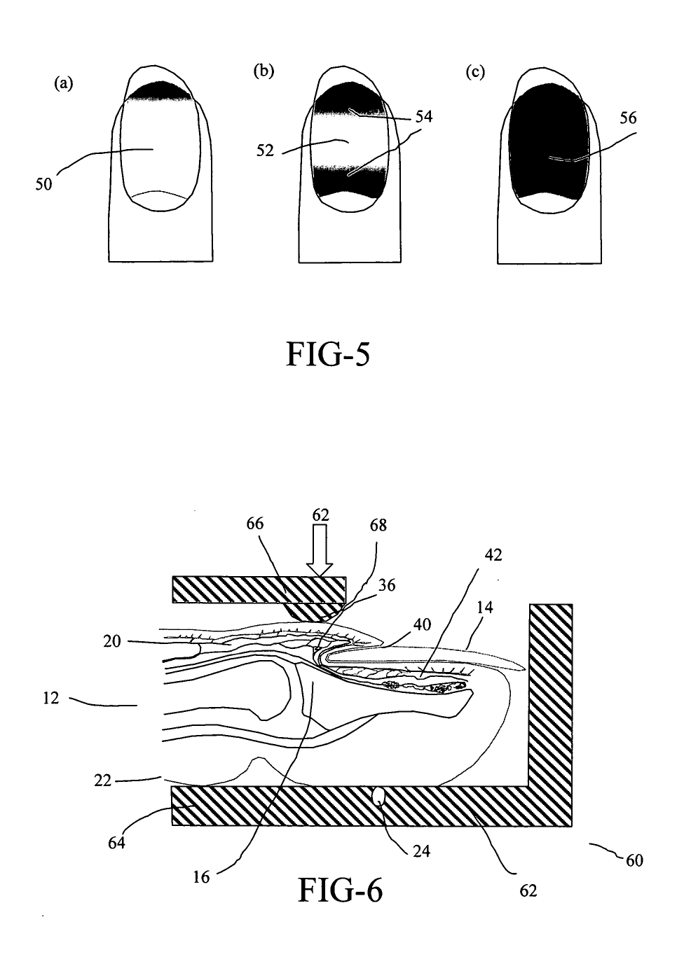

The cartoon in FIG. 5(a) illustrates the intended effect of a finger holder such as, for example, that shown in FIG. 4. The finger represented in FIG. 5(a) has a white region 50 in which blood has been largely excluded from the area under the nail by pressing the nail back along the axis of the finger as described above. In comparison, a fingernail with no pressure exerted upon it is represented by FIG. 5(b) in which a lighter central region 52 is surrounded by darker blood-rich regions 54. A finger holder such as that depicted in FIG. 4 is thus well suited for Raman spectroscopy in the blue, UVA and visible. The sterile matrix 42 beneath the nail 14 contains interstitial fluid containing glucose in a concentration similar to that of the blood stream. When the fingernail plate is pushed back into the fingertip bone as described above, it suppresses the arterial vessels lying in the narrow region behind the fingernail's root and the bone. As a result, it suppresses the blood supply to the sterile matrix under the nail plate. In this manner, blood may be largely excluded from the sterile matrix, so the interference of the strong absorbance of hemoglobin in these spectral regions with both the incident excitation light and Raman scattered radiation is substantially reduced.

Incident light in the blue spectral region generally and more specifically at a wavelength of approximately 480 nm and alternatively in the UVA spectral region generally and more specifically at a wavelength of approximately 370 nm has a relatively good spectral window to probe the interstitial fluid in the sterile matrix under a nail wherein blood hemoglobin is substantially excluded. A system and method according to this embodiment offers substantial benefits over previously available spectroscopy-based in vivo analysis methods. Use of an excitation wavelength in the blue or UVA results in a dramatically increased Raman cross-section. Measurement of the anti-Stokes Raman spectrum either in addition to or in lieu of the Stokes spectrum permits avoidance of much of the fluorescence background that may hinder accurate determination of analyte concentrations based solely on Stokes Raman emissions. Tissue that is perfused with mostly interstitial fluid and little blood permits light at these wavelengths to penetrate more deeply, thereby resulting in a longer path length and an increased Raman signal.

7. Red and NIR Embodiment

In another embodiment of the present invention, a tissue containing both interstitial fluid and vascular fluid is probed. Use of red and NIR wavelengths for the incident excitation light may allow the total extracellular fluid in the sampled volume of the sterile matrix to be increased, thereby improving the Raman signal intensity. In this embodiment, a finger (or toe) holder such as that shown schematically in FIG. 6 may be used to encourage blood pooling under the nail. FIG. 6 depicts a finger 12 having a nail plate 14, fingertip bone 16, blood vessels 20, skin 22, arterial vessels 36 lying between the subcutaneous end 40 of the nail plate 14 and the fingertip bone 16, and the sterile matrix 42. The finger holder 60 according to this embodiment also comprises a sensor 24 to measure finger temperature through contact with the finger. According to this embodiment, the finger 12 is pressed down in the direction of the arrow 62 to the base plate 64 by a pressure arm 66 that may advantageously include a touch pad 68. The touch pad 68 may be formed of a resilient material that does not discomfort the finger but still applies sufficient pressure to hold it stationary. This arrangement can be adjusted to provide a level of force on the fingertip that provides the maximal amount of blood pooling in the sterile matrix. Pressure may be applied in the range of approximately 1 to 4 Newtons. The pressure from both top and bottom will temporarily suppress the digital vascular blood flow, thereby causing the sterile matrix to be in the blood replete state.

During the blood pooling, pulse-caused fluctuations may also be minimized. Although a patient may simply press his/her finger down on a flat surface to cause the sterile matrix to become blood replete, use of suitable clamp means such as the pressure arm 66 is advantageous to provide consistent and uniform downward pressure and maintain the finger stationary. The holder of FIG. 6 provides enhanced and steadier blood pooling than simply pressing the finger down. Therefore, such a finger holder not only holds the finger in place, but also creates an ideal situation for blood pooling. After clamping down, the finger holder may, if desired, be traversed to optimize the alignment of the fingernail sterile matrix with the focus of the laser beam and the focus of the parabolic mirror. Alternatively, the illumination and collection optical system may be translated instead of moving the finger holder, which may remain stationary.

As noted above, pressing of a digit 12 downward onto a fixed surface has the effect of causing blood to pool in the sterile matrix 42 beneath the nail 14. As noted above, red and/or NIR excitation wavelengths do not coincide with the strong absorbance regions of the hemoglobin spectrum shown in FIG. 1. Thus, an increase in the amount of blood in the sample volume within the sterile matrix 42 increases the concentration of glucose and/or other analytes of potential interest in the sample volume without negatively impacting the intensity of incident light entering the sample or the scattered radiation leaving the sample. The intensity of the scattered Raman radiation to be measured by the analysis system is thereby increased. The cartoon of a finger shown in FIG. 5(c) illustrates the effect of downward pressure on blood supply in the sterile matrix. As shown, the nail 56 is more uniformly dark compared to the finger at rest as shown in FIG. 5(b).

The laser or other excitation light source for anti-Stokes Raman analysis according to the this embodiment advantageously has a wavelength in the range of approximately 600 nm to 980 nm. This wavelength regime results in a very good spectral window in the tissue even when the tissue is largely perfused with blood containing hemoglobin. The lower end of the advantageous wavelength range is at just slightly higher wavelength than the second strong absorption peak of hemoglobin, and the upper end of the range approaches the detection limit for currently available CCDs. Further developments in CCD technology may allow use of wavelengths above the recited upper end of the wavelength range.

8. Probe and Analysis Systems and Methods

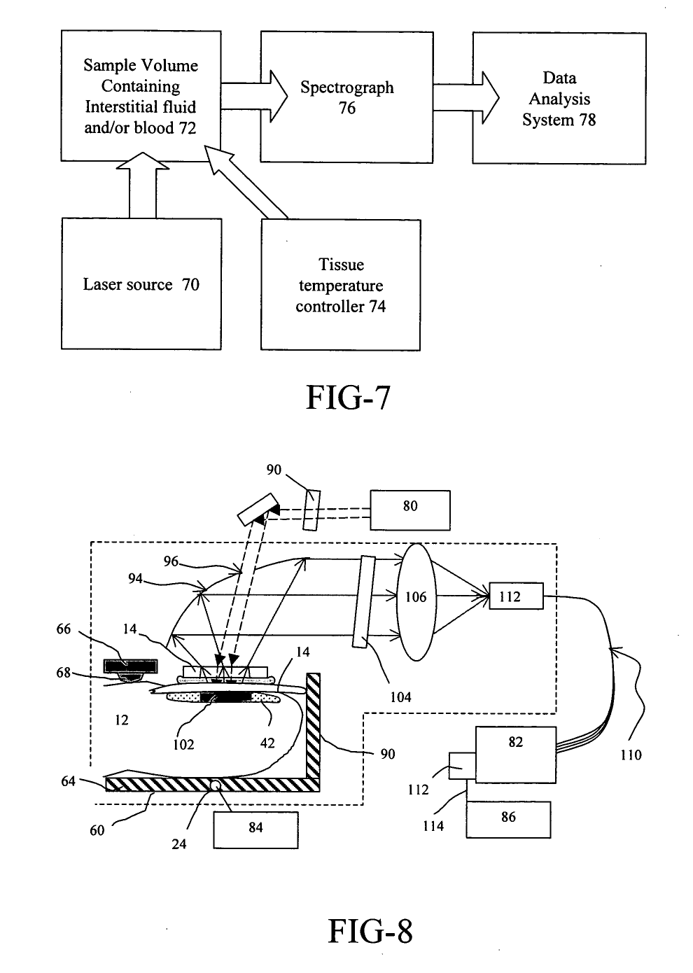

In general, a system for Raman analysis according to the present invention may be represented functionally as shown in FIG. 7. A laser source illuminates 70 a sample volume containing interstitial fluid and/or interstitial fluid and blood 72. A tissue temperature controller system 74 monitors and optionally provides heat to a finger or toe in response to the difference between the digit and a preferred temperature which may be body temperature. Light waves scattered within the sample volume are collected by an optics system and transmitted to a spectrograph 76 wherein intensity response is quantified as a function of wavelength. Data from the spectrograph are provided to a spectral analysis system 78 that processes the data using partial least squares and a Boltzmann exponential factor correction to account for the temperature of the sample volume during data collection.

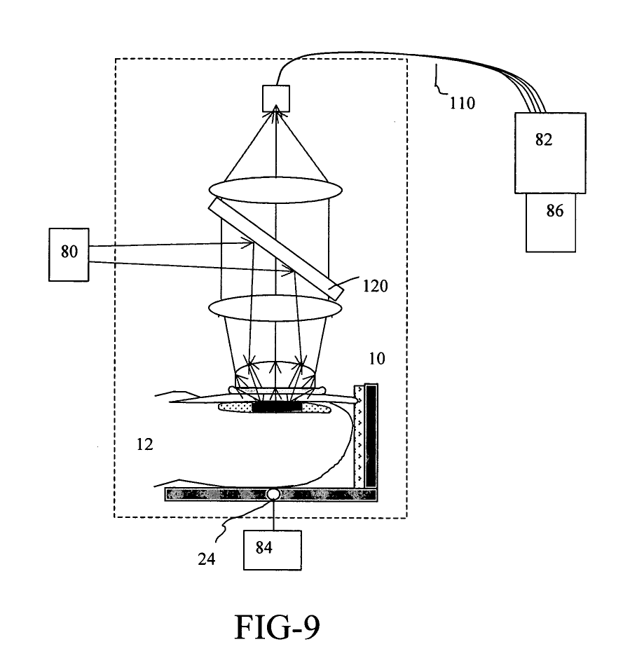

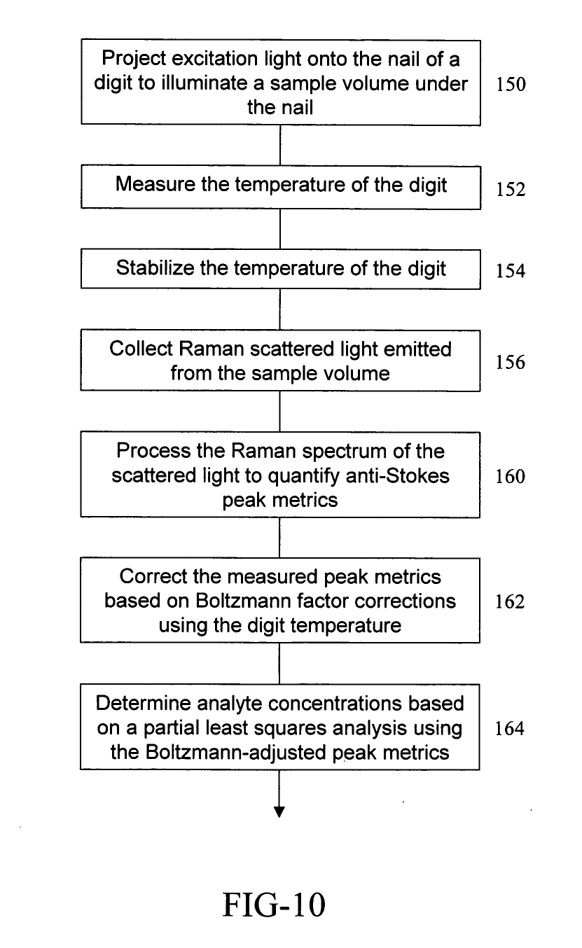

In more detailed exemplary embodiments of the present invention, systems and methods are provided for probing tissue containing predominantly interstitial fluid. The optical probe projects a laser beam onto the tissue under a nail and collects anti-Stokes Raman light from the tissue. As illustrated in FIG. 8 and FIG. 9, systems according to the present invention generally comprise a laser or comparable collimated, single wavelength excitation light source 80, optical components to deliver the excitation light to and collect light scattered from the sampled tissue, a spectrograph 82, a tissue temperature monitor and stabilizer 84, and a computer 86 to perform a PLS type multi-variate regression analysis procedure including the Boltzmann factor.

Referring more specifically to FIG. 8, one embodiment of a sampling system is shown for use with the red/NIR embodiments described above. In this example, a finger is placed in a finger holder 60 such as illustrated in greater detail in FIG. 6. A beam of light from a diode laser or other suitable source of collimated, single wavelength excitation light 80 is passed through a bandpass filter 90 and then passed through a parabolic mirror 94 by means of a small hole 96 in the mirror, and is focused onto a nail 14 optionally adapted with a gel window 100. Under the nail 14, a blood sample from the blood rich capillaries in the sterile matrix 42 is pooled under pressure. A sample volume 102 within the sterile matrix 42 is thus illuminated with excitation light. The excitation light source 80 may provide light with a wavelength in the range of approximately 600 to 980 nm or advantageously at approximately 830 nm. Examples of potential embodiments of the gel-adapted window 100 are described in greater detail in co-pending U.S. patent application Ser. No. 10/723,042, the disclosure of which has been incorporated in its entirely.

Raman-scattered light emitted from blood in the sample volume 102, which may have a cross sectional area of approximately 1 mm², is collected by the mirror 94, passed through a notch filter 104 configured to reject light at the excitation light wavelength, and then focused by a lens 106 into an optical fiber bundle 110. The optical fiber bundle 110 may optionally be fitted with an input orifice 112 that converts the circular shape of the collected light to a rectangular shape to match the entrance slit of a spectrograph 82. The spectra are collected by a cooled charge coupled device (CCD) array detector 112, in this example a CCD array detector having 1024×256 pixels, and binned along the vertical direction, resulting in a 1024 pixel spectrum.

Additional examples of alternative probes that may be used in conjunction with this embodiment of the present invention are described in greater detail and illustrated in FIG. 12 and FIG. 13 of co-pending U.S. patent application Ser. No. 10/723,042. For use with the red/IR embodiment as described above, these probes may advantageously include a finger holder 60 comprising a base surface 64 against which a finger 12 (or toe) is pressed downward to encourage blood pooling in the sterile matrix 42 beneath the nail 14. The finger holder 60 further comprises a temperature sensor 24 and temperature stabilization means 84. The temperature stabilization means may involve a feedback loop to a data processor that records the current temperature of the finger (or toe) in the holder 60 and reactively powers one or more heating elements to raise and/or stabilize the finger (or toe) temperature as needed to maintain a constant, known temperature in the sample volume 102. One of ordinary skill in the art may also readily understand that any of the probes described above may be modified for use with the blue/UV embodiment as described in greater detail below through the substitution of a finger holder such as that shown in FIG. 4 and substitution of a excitation light source with the appropriate wavelength.

FIG. 9 illustrates one possible probe system for use with the blue/UV embodiment described above. In general, the probe in FIG. 9 comprises a finger holder 12 similar to that shown in FIG. 4, a laser beam or other collimated, single-wavelength excitation light source 80 that is focused onto the sterile matrix 42 beneath the nail 14 of a finger 12 (or toe) and collection optics for the resulting Raman scattered radiation. The excitation laser has a wavelength that may advantageously be in the blue or UV spectral region generally and advantageously approximately 480 nm or approximately 370 nm. As noted above, one of ordinary skill in the art will understand that other wavelengths may be used based on routine experimentation using the teachings provided herein. The finger holder 10 optionally further comprises a temperature monitor 24 and a means for monitoring and stabilizing the finger temperature 84.

Referring more specifically to FIG. 9, the excitation light beam from the light source 80 passes through a dichroic beam splitter 120 having high transmission. Raman light collected from the sterile matrix is reflected by the beam splitter because it is at a different wavelength from the incident laser light. The reflected Raman scattered light is then coupled into a spectrometer 82 to record the Raman spectrum. In this as well as the above-described probe embodiments, the spectrograph 82 may further comprise a linear array of fibers forming a fiber bundle from the probe at the entrance, a grating for dispersing the spectrum (not shown), a CCD detector 112 for collecting and processing the spectrographic image, and a connection 114 between the CCD 112 and a computer 86 for data acquisition and processing.

As noted above for the red/IR probe, a tissue temperature monitor 24 and temperature stabilizer means 84 may be implemented in the finger holder 10, 60 to monitor the temperature of the finger 12 (or toe) and provide a higher thermal mass to stabilize the temperature. If the finger is too cold, the system may be configured with a feedback loop and warning signal to indicate that the patient should warm the finger before a measurement is taken. Alternatively, the finger holder 10, 60 may be implemented with a heating element (not shown) coupled via a feedback loop to a temperature controller receiving input from the temperature monitor 24 to warm and stabilize the finger at a known, constant temperature that is near normal human body temperature. Anti-Stokes Raman measurements are advantageously not made until the sample volume reaches the stable target temperature.

The anti-Stokes Raman spectrum may be collected from the tissue and analytes contained within the tissue using a probe according to the present invention. Potential analytes may include, but are not limited to, glucose, urea, cholesterol, triglycerides, total protein, albumin, hemoglobin, hematocrit, and bilirubin and other analytes in interstitial fluid and blood as well as those in the cell. Use of a PLS type multi-variate regression analysis procedure including a Boltzmann calibration function may advantageously disentangle the spectra to yield glucose and/or other relevant analyte concentrations.

The red/NIR embodiment offers substantial benefits for blood rich tissues. It also permits a longer path length in the tissue. As a result, it increases the total Raman signal and helps overcome the low cross section. In addition, use of the anti-Stokes Raman spectrum with red and/or IR wavelength excitation eliminates the fluorescence background that interferes with Stokes Raman signals. The overall signal to noise ratio is improved quite significantly. The blue/UV embodiment offer substantial advantages in improved anti-Stokes Raman response due to the higher energy of the incident photons. Although the fluorescence response from the sample tissue may also be increased by use of higher energy photon, as noted above, anti-Stokes Raman emissions generally occur in a different spectral region than fluorescence emissions.

One embodiment of a method of Raman anti-stokes analysis of blood analytes according to the present invention is summarized in the flow chart shown in FIG. 10. Referring to FIG. 10, the concentration of an analyte in blood or another body fluid may be determined in vivo without the need to draw blood. A beam of excitation light is projected by an optics system or alternatively directly from the light source onto a finger or toe nail 150. As described above, the digit may be positioned within a holder that measures 152 and/or stabilizes 154 the temperature of the digit prior to analysis. The beam of excitation light shines through the nail to the sterile matrix beneath the nail and elicits a Raman spectrum having Stokes and anti-Stokes regions. Raman scattered light emitted within the sample volume of the sterile matrix is collected by an optics system 156. The collected light may be transmitted to a spectrometer optionally including a charge coupled detector (CCD) or some other detector that processes the incoming Raman spectrum to quantify the peak metrics of anti-Stokes radiation emitted in the sample volume 160. These peak metrics may include peak height, peak area, or other measures of the light intensity at a given wavelength. The measured peak metrics are then corrected using a Boltzmann factor 162 that is based on the measured and/or stabilized temperature of the digit to account for variations in the population of molecules in the excited energy states necessary to emit anti-Stokes radiation. Finally, analyte concentrations are calculated based on a partial least squares analysis of the peak metrics using the Boltzmann-adjusted peak metrics 164.

The foregoing description of specific embodiments and examples of the invention have been presented for the purpose of illustration and description, and although the invention has been illustrated by certain of the preceding examples, it is not to be construed as being limited thereby. They are not intended to be exhaustive or to limit the invention to the precise forms disclosed, and obviously many modifications, embodiments, and variations are possible in light of the above teaching. It is intended that the scope of the invention encompass the generic area as herein disclosed, and by the claims appended hereto and their equivalents.

1. A method for in vivo detection of an analyte, comprising the steps of:
illuminating a sample volume of body tissue with a beam of optical radiation at an incident wavelength from an optical source;
collecting scattered anti-Stokes Raman radiation emitted from within the sample volume;
analyzing the collected scattered anti-Stokes Raman radiation to determine a intensity response as a function of wavelength;
calculating the analyte concentration based on the intensity response as a function of wavelength.

2. The method of claim 1, wherein the sample volume lies within a sterile matrix beneath a nail and the beam of incident optical radiation passes through the nail to illuminate the sample volume.

3. The method of claim 1, wherein the analyte concentration is calculated using a partial least squares method.

4. The method of claim 1, further comprising the steps of:
measuring and/or stabilizing the temperature of the sample volume prior to collecting and analyzing the scattered anti-Stokes Raman radiation.

5. The method of claim 4, further comprising the step of applying a Bolztmann correction factor to adjust the intensity response as a function of wavelength, wherein the Bolztmann correction factor is a function of the measured and/or stabilized temperature of the sample volume.

6. The method of claim 1, wherein the incident wavelength is in the red or near-infrared region of the electromagnetic spectrum.

7. The method of claim 6, further comprising the step of:
pressing a digit having a nail downward onto a fixed surface such that blood pools in a sterile matrix beneath the nail, wherein the beam of incident optical radiation passes through the nail to illuminate the sample volume.

8. The method of claim 7, wherein the incident wavelength is in the range of approximately 600 nm to 980 nm.

9. A system for implementing the method of claim 7, comprising:
a digit holder that comprises a fixed surface onto which the digit may be pressed downward;
a source of incident optical radiation providing light at the incident wavelength;
a spectrometer; and
a data processing system that executes a software routine that calculates the analyte concentration based on the intensity response as a function of wavelength.

10. The method of claim 1, wherein the incident wavelength is in the blue or ultraviolet region of the electromagnetic spectrum.

11. The method of claim 10, further comprising the step of:
pressing a digit having a nail forward into a fixed surface such that the nail is compressed back into the finger, thereby restricting the flow of blood into a sterile matrix beneath the nail, wherein the beam of incident optical radiation passes through the nail to illuminate the sample volume.

12. The method of claim 11, wherein the incident wavelength is approximately 370 nm.

13. The method of claim 11, wherein the incident wavelength is approximately 480 nm.

14. A system for implementing the method of claim 11, comprising:
a digit holder that comprises a fixed surface into which the digit may be pressed forward to compress the nail back into the digit;
a source of incident optical radiation providing light at the incident wavelength;
a spectrometer; and
a data processing system that executes a software routine that calculates the analyte concentration based on the intensity response as a function of wavelength.

15. A system for using anti-Stokes Raman spectography to detect an analyte in vivo, comprising:
a digit holder for positioning a digit, the digit comprising skin and a nail plate, the nail plate having a first end that is under the skin and a second opposite end disposed proximate to a tip of the digit, the digit holder comprising a substantially flat base plate attached to a back wall, the back wall being disposed approximately perpendicularly to the base plate, such that the digit may be placed in the holder with a side of the digit opposite to the nail plate resting on the base plate and the second end of the nail plate disposed proximate to the back wall;
a sensor for measuring the temperature of the digit, the sensor being attached to the digit holder;
an incident light source, the incident light source providing excitation radiation at an excitation wavelength, the excitation radiation being directed through the nail plate into a sterile matrix beneath the nail plate; and
a collection subsystem, the collection subsystem receiving scattered radiation emitted within the sterile matrix.

16. The system of claim 15, further comprising:
an optics system, the optics system directing the excitation radiation to the nail plate, the optics system further directing scattered radiation emitted from the sterile matrix in response to the excitation radiation to the collection system.

17. The system of claim 15, wherein:
a surface of the back wall is formed of a firm, padded material such that the digit may be comfortably pressed toward the back wall to compress the nail plate back into the finger to suppress blood flow into the sterile matrix; and
the excitation wavelength is in the blue region of the spectrum.

18. The system of claim 17, wherein the excitation wavelength is approximately 370 nm.

19. The system of claim 17, wherein the excitation wavelength is approximately 480 nm.

20. The system of claim 15, wherein:
the digit holder further comprises a pressure arm for pressing and holding the digit against the base plate; and
the excitation wavelength is in the range of approximately 600 nm to 980 nm.

21. The system of claim 15, further comprising:
a heating element attached to the digit holder; and
a data processor, the data processor receiving temperature data from the sensor and reactively powering the heating element to raise and/or stabilize the temperature of the digit.

22. The system of claim 15, further comprising:
a gel-adapted window, the gel adapted window being placed on the nail plate to provide a uniform optical interface through which the excitation radiation and the scattered radiation may pass.

23. A method for in vivo detection of an analyte, comprising the steps of:
projecting excitation light onto a nail of a digit to illuminate a sample volume under the nail;
measuring the temperature of the digit;
collecting Raman scattered light emitted from the sample volume, the Raman scattered light comprising an anti-Stokes signal;
processing the Raman spectrum of the scattered light to quantify one or more peak metrics for the anti-Stokes signal;
correcting the peak metrics based on a Boltzmann correction factor, the Boltzmann correction factor being calculated using the measured temperature of the digit; and
determining the concentration of the analyte based on a partial least squares analysis using the Boltzmann-adjusted peak metrics.

24. The method of claim 23, further comprising the step of stabilizing the temperature of the digit.
